(19) United States
(12) Patent Application Publication
(10) Pub. No.: US 2008/0286251 A1
(43) Pub. Date: Nov. 20, 2008

(54) Compositions and Methods for the Enhancement of Chemotherapy with Microbial Cytotoxins

(75) Inventors: David Platt, Newton, MA (US); Eliezer Zomer, Newton, MA (US)

Correspondence Address: GREENBERG TRAURIG, LLP, 200 PARK AVE., P.O. BOX 677, FLORHAM PARK, NJ, 07932, US

(73) Assignee: PROPHARMACEUTICALS, INC., Newton, MA (US)

(21) Appl. No.: 11/659,170

(22) PCT Filed: Aug. 2, 2005
(86) PCT No.: PCT/US05/27187

§ 371 Date: May 14, 2008

Related U.S. Application Data
(60) Provisional application No. 60/598,176, filed on Aug. 2, 2004.

(51) Int. Cl. A61K 35/74; A61P 35/00

(52) U.S. Cl. 424/93.41; 424/93.1; 424/93.4

(57) ABSTRACT

Described herein is a microbial composition used to enhance anti-cancer drugs. Specifically, microbial compositions that comprise a part of or an entire microorganism having surface lectins specific to carbohydrate moieties on tumor surface combined with an oncolytic agent.

### RELATED APPLICATIONS

This application claims the benefit of and priority to U.S. Provisional Patent Application Ser. No. 60/598,176, filed Aug. 2, 2004.

### FIELD OF THE INVENTION

The present invention pertains to the use of a microbial composition to enhance anti-cancer drugs. Specifically, the instant invention relates to microbial compositions that comprise a part of or an entire microorganism having surface lectins specific to carbohydrate moieties on tumor surface.

### BACKGROUND OF THE INVENTION

An historical review reveals a number of clinical observations in which cancers were reported to regress in patients with bacterial infections. Nauts et al., 1953, Acta Medica. Scandinavica 145:1-102, (Suppl. 276) state: Shear, 1950, J.A.M.A. 142:383-390 (Shear), observed that 75% of the spontaneous remissions in untreated leukemia in the Children's Hospital in Boston occurred following an acute episode of bacterial infection.

Subsequent evidence from a number of research laboratories indicated that at least some of the anti-cancer effects are mediated through stimulation of the host immune system, resulting in enhanced immuno-rejection of the cancer cells. For example, release of the lipopolysaccharide (LPS) endotoxin by Gram negative bacteria such as Salmonella triggers release of tumor necrosis factor, TNF, by cells of the host immune system, such as macrophages. Elevated TNF levels in turn initiate a cascade of cytokine-mediated reactions which culminate in the death of tumor cells.

As a result of such observations as described above, immunization of cancer patients with BCG injections is currently utilized in some cancer therapy protocols. See Sosnowski, 1994, Compr. Ther. 20:695-701; Barth and Morton, 1995, Cancer 75 (Suppl. 2):726-734; Friberg, 1993, Med. Oncol. Tumor. Pharmacother. 10:31-36 for reviews of BCG therapy.

Pan et al. in 1995, Nature Medicine 1:471-477, described the use of Listeria monocytogenes as a vaccine for the immunization of mice against lethal challenges with tumor cells expressing the same antigen expressed by the Listeria vaccine. In addition, they showed regression of established tumors when immunized after tumor development in an antigen specific T-cell-dependent manner. However, Pan et al. failed to show that Listeria monocytogenes could be used as a tumor specific vector, which would target and amplify within the tumor.

Pawelek, et al. U.S. Pat. No. 6,190,657 teach the use of super-infective, tumor-specific, attenuated strains of parasites as an anti-cancer composition including Salmonella spp., Mycobacterium avium, or the protozoan Leishmania amazonensis, all potential pathogens which can cause lethal diseases.

The use of Salmonella has been discussed in numerous papers. However, the risks of pathogenicity of salmonella always outweigh the potential benefit and efforts have been to attenuate the strains to reduce toxicity. For example Carrier et al., 1996, discuss the “Expression of human IL-1β in Salmonella typhimurium; a model system for the delivery of recombinant therapeutic proteins in vivo”, J. Immunology 148:1176-1181. Chabalgoity et al., 1996, “A Salmonella typhimurium htrA live vaccine expressing multiple copies of a peptide comprising amino acids 8-23 of herpes simplex virus glycoprotein D as a genetic fusion to tetanus toxin fragment C protects mice from herpes simplex virus infection”, Microbiol. 19:791-801.

Clostridium was previously investigated as a potential therapeutic vector for solid tumors. The propensity of spores of the obligate anaerobe Clostridium to germinate in necrotic tissues is well known. Tetanus and gas gangrene result from successful colonization of necrotic tissue by pathogenic members of this genus. See: Fox, et al., 1996, “Anaerobic bacteria as a delivery system for cancer gene therapy: in vitro activation of 5-fluorocytosine by genetically engineered Clostridia”, Gene Therapy 3:173-178. S. Friberg, 1993, “BCG in the treatment of superficial cancer of the bladder: A review”, Med Oncol Tumor Pharmacother 10:31-36. J. Galan, 1995, “Novel salmonella antigen delivery vectors”, NIH project No. 5 R01 AI36520-02. Gericke and Engelbart, 1963, “Oncolysis by Clostridia. II.

Sizemore et al., in 1995, Science 270:299-302, describe the use of attenuated Shigella bacteria as a DNA delivery vehicle for DNA-mediated immunization. Sizemore et al. showed that an attenuated strain of Shigella invaded mammalian cells in culture and delivered DNA plasmids containing foreign genes to the cytoplasm of the cells. Foreign protein was produced in mammalian cells as a result of the procedure.

Parker et al., 1947, Proc. Soc. Exp. Biol. Med. pp. 461-467 first showed that direct injection of spores of Clostridium histolyticus into a transplantable sarcoma growing in a mouse caused oncolysis, i.e., liquification, as well as regression of the tumor. In general, the process of Clostridium-mediated oncolysis was accompanied by acute toxicity and death of the mice. Malmgren and Flanigan, 1955, Cancer Res. 15:473 demonstrated that mice bearing mammary carcinomas, hepatomas, and other tumors died within 48 hrs of intravenous injection of Clostridium tetani spores, whereas control, non-tumor bearing animals were asymptomatic for 40 days.

Thiele et al., 1964, Cancer Res. 24:222-233 showed that intravenously injected spores of a number of species of nonpathogenic Clostridia, including M-55, localized and germinated in tumor tissue, but not in normal tissues of the mouse. Thiel et al., 1964, Cancer Res. 24:234-238 found that spore treatment produced no effect when administered early in the development of the tumor, i.e., when the tumors were of small size. While the spores caused oncolysis in tumors of sufficient size, there was no effect in smaller tumors or metastases. The animals regularly died during oncolysis. Carey et al., 1967, Eur. J. Cancer 3:37-46, concluded that small tumors and metastases had been noted to be resistant to oncolysis whereas large neoplasms were particularly favorable. Thus, the qualitative differences in germination of spores were likely not to be a characteristic of neoplastic and normal tissues per se, but related to physiologic and biochemical conditions found within large tumor masses.

Nothing in any of the above references (or any other references known to the present inventors) suggests the combination of cytotoxic microorganism with chemotherapeutics to fight solid tumors. Furthermore, none of the studies and patents identify cytotoxic microorganism that contain lectins which target carbohydrate on metastatic tumor cells and can deliver cytotoxic material to cause necrosis and enhance chemotherapeutic drugs. The present invention addresses these deficiencies.

### BRIEF SUMMARY OF THE INVENTION

The present invention pertains to the use of a microbial composition to enhance anti-cancer drugs. Specifically, the instant invention relates to microbial compositions that comprise a part of or an entire microorganism having surface lectins specific to carbohydrate moieties on tumor surface.

One embodiment the invention is directed to a composition employed for the treatment of a subject with a cancerous tumor including those that metastasize, comprising the co-administration of an effective amount of a cytotoxic microorganism and a chemotherapeutic drug to the subject. In one aspect, this microorganism, or a derivative thereof, comprises lectins which bind to carbohydrate moieties on a tumor surface which enhances the chemotherapeutic drug and synergistically inhibits tumor growth (including metastasis) by, e.g., effectuating tumor necrosis thereby resulting in tumor death.

Another embodiment of the present invention is directed toward a composition used for the treatment of a subject with a cancerous tumor and metastasis, comprising the co-administration of an effective amount of a cytotoxic fixed microorganism capsules and a chemotherapeutic drug. In one aspect, the microorganism comprises surface lectins which bind to one or more carbohydrate moieties on the tumor surface and combined with chemotherapeutic drugs, synergistically inhibits tumor growth (including metastasis) by disrupting and causing tumor necrosis and further causing tumor death with the chemotherapeutics. In one aspect, the surface carbohydrate is a galectin. In a particular aspect, the surface carbohydrate is galectin-3.

A method of treating a subject suffering from cancer by administering an therapeutic effective amount of a cytotoxic microorganism and one or more chemotherapeutic agents. In one aspect, the microorganism is attenutated using a method known to those of skill in the art. In another aspect, the microorganism is a fixed microorganism capsule. In this method, the microorganism comprises lectins capable of binding to surface receptors present on cancer cells. In one aspect, the surface receptors are galectins. In a particular aspect, the receptors are galectin-3 receptors.

For a better understanding of the present invention, together with other and further objects thereof, reference is made to the accompanying drawings and detailed description and its scope will be pointed out in the appended claims.

### BRIEF DESCRIPTION OF THE SEVERAL VIEWS OF THE DRAWING

FIG. 1 is a graph of the effect of B. pertussis alone and in combination with 5-Fluorouricil and galactomannan therapy on melanoma B-16 in mice; and

FIG. 2 is a bar graph of the effect of B. pertussis alone and in combination with 5-Fluorouricil and galactomannan therapy on melanoma B-16 in mice.

### DETAILED DESCRIPTION OF THE INVENTION

The present invention pertains to the use of a microbial composition used to enhance anti-cancer drugs. Specifically, the instant invention relates to microbial compositions that comprise a part of or an entire microorganism having surface lectins specific to carbohydrate moieties on tumor surface.

“Administration” refers to oral, or parenteral including intravenous, subcutaneous, topical, transdermal, transmucosal, intraperitoneal, and intramuscular.

“Subject” refers to an animal such as a mammal, for example, a human. The term also includes patients.

“Treatment of cancer” refers to prognostic treatment of subjects at high risk of developing a cancer as well as subjects who have already developed a tumor. The term “treatment” can be applied to the reduction or prevention of abnormal cell proliferation, cell aggregation and cell dispersal (metastasis) to secondary sites.

“Cancer” refers to any neoplastic disorder, including such cellular disorders as, for example, renal cell cancer, Kaposi's sarcoma, chronic leukemia, breast cancer, sarcoma, ovarian carcinoma, rectal cancer, throat cancer, melanoma, colon cancer, bladder cancer, mastocytoma, lung cancer, mammary adenocarcinoma, pharyngeal squamous cell carcinoma, and gastrointestinal or stomach cancer.

“Effective dose” refers to a dose of an agent that improves the symptoms of a subject or the longevity of the subject suffering from or at high risk of suffering from cancer.

“Anti-cancer drug” refers to, within the context of this application, any of a variety of compounds which exhibit efficacy in reducing the size, incidence, metastasis, proliferation, occurrence, or recurrence of cancer tumors or tumor cells, including, but not limited to: aminoglutethimide, Amsacrine, Anastrozole, asparaginase, BCG, bicalutamide, Bleomycin, Buserelin, Busulfan, Capecitabine, carboplatin, Carmustine, chlorambucil, cisplatin, Cladribine, Clodronate, cyclophosphamide, cyproterone, Cytarabine, Dacarbazine, Dactinomycin, Daunorubicin, diethylstilbestrol, Docetaxel, Doxorubicin, Epirubicin, Estramustine, etoposide, Exemestane, Filgrastim, Fludarabine, Fludrocortisone, fluorouracil, Fluoxymesterone, Flutamide, Gemcitabine, Goserelin, hydroxyurea, Idarubicin, Ifosfamide, Imatinib, Interferon Alfa, Irinotecan, Letrozole, Leucovorin, Leuprolide, Levamisole, Lomustine, Mechlorethamine, Medroxyprogesterone, Megestrol, Melphalan, mercaptopurine, Mesna, methotrexate, mitomycin, Mitotane, Mitoxantrone, Nilutamide, Octreotide, Oxaliplatin, Paclitaxel, Pamidronate, Pentostatin, Plicamycin, Porfimer, procarbazine, Raltitrexed, Rituximab, streptozocin, Tamoxifen, Temozolomide, Teniposide, testosterone, thioguanine, Thiotepa, Topotecan, Trastuzumab, Tretinoin, Vinblastine, Vincristine, Vindesine, Vinorelbine, and the like.

The problems associated with the physical barriers associated with target delivery of therapeutic agents to solid tumors provide clear and difficult obstacles in the design of an effective delivery system. Thus, there has been a long felt need in the art to provide delivery systems which are able to overcome these obstacles.

We have shown that lectin specific polysaccharides can enhance chemotherapeutic activity against solid tumors (Klyosov and Platt U.S. Pat. No. 6,645,946, the entire teaching of which is incorporated herein by reference). In the present invention, we have combined chemotherapy drugs with cytotoxic microorganisms which posses lectins that have the ability to bind to cancer cells and cause degradation of solid tumor integrity and facilitates therapeutic drugs to increase effectiveness. The methods described herein do not require a live vector or multiplication at the cancer site as required by methods described in the prior art. On the contrary, the use of microoraganisms like B. pertussis, which most people have been immunized against, will further reduce any risk associated with secondary infections by the pathogen. We further use a two treatment sequence, first the administration of a cytotoxic live microbial agent, followed by the administration chemotherapeutic drug to elicit a therapeutic effect. (Contrary to the prior art, the amplification capacity under either aerobic or anaerobic conditions is not an advantage or pre-requisite for effective anti tumor-activity.) The methods described herein also are directed toward the use of attenuated microorganisms. One skilled in the art well appreciates the various methods that can be employed to attenuate a microbe, such as heat, use of a caustic agent, genetic engineering and the like.

In one embodiment, a composition is formulated for the treatment of a tumorous cancer and metastasis, comprising a cytotoxic microorganism, which contains lectins that bind to one or more carbohydrate moieties on the surface of the tumor cells which enhances the therapeutic effect of one or more chemotherapeutic drugs by synergistically inhibiting tumor growth. In one aspect, the lectin interacts with a galectin receptor on the cancer cell. In a particular aspect, the galectin is galectin-3. One mechanism of action posited is that this combination disrupts and effectuates tumor necrosis.

In another embodiment, the composition for treating a tumor cancer and metastasis, comprises an effective amount of cytotoxic fixated microorganism capsules, which contain surface lectins that bind to carbohydrate moieties on the surface of a tumor cell used in combination with one or more chemotherapeutic drugs, wherein this combination synergistically inhibits tumor growth by disrupting and causing tumor necrosis.

Another embodiment of the invention is directed to a composition employed for the treatment of a subject having a cancerous tumor and metastasis, comprising the co-administration of an effective amount of a cytotoxic microorganisms and a chemotherapeutic drug to the subject. In one aspect, this microorganism comprises lectins which bind to carbohydrate moieties on the surface of a tumor cell, wherein this combination enhances the chemotherapeutic drug and synergistically inhibits tumor growth by effectuating tumor necrosis thereby resulting in tumor death.

In another embodiment, a composition is formulated in accordance with what is well appreciated by those skilled regarding pharmaceutical compositions adapted for intravenous (i.v.) or intraperitoneal (i.p.) administration to human beings. Typically, compositions for i.v. and i.p. administration are solutions in sterile isotonic aqueous buffer. Where necessary, the composition may also include a stabilizing agent and a local anesthetic such as lidocaine to ease the pain at the site of the injection. Generally, the components are supplied either separately or mixed together in unit dosage form, e.g., as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Any of the identified compounds of the present invention can be administered to a subject, including a human, by itself, or in pharmaceutical compositions where it is mixed with suitable carriers or excipients at doses therapeutically effective to prevent, treat or ameliorate a variety of disorders, including those characterized by that outlined herein. A therapeutically effective dose further refers to that amount of the compound sufficient result in the prevention or amelioration of symptoms associated with such disorders. Techniques for formulation and administration of the compounds of the instant invention may be found in Goodman and Gilman's The Pharmacological Basis of Therapeutics, Pergamon Press, latest edition.

The compounds of the present invention can be targeted to specific sites by direct injection into those sites. Compounds designed for use in the central nervous system should be able to cross the blood-brain barrier or be suitable for administration by localized injection.

Pharmaceutical compositions suitable for use in the present invention include compositions wherein the active ingredients are contained in an effective amount to achieve its intended purpose. More specifically, a therapeutically effective amount means an amount effective to prevent development of or alleviate the existing symptoms and underlying pathology of the subject being treating. Determination of the effective amounts is well within the capability of those skilled in the art.

For any compound used in the methods of the present invention, the therapeutically effective dose can be estimated initially from cell culture assays. For example, a dose can be formulated in animal models to achieve a circulating concentration range that includes the IC₅₀ (the dose where 50% of the cells show the desired effects) as determined in cell culture. Such information can be used to more accurately determine useful doses in humans.

A therapeutically effective dose refers to that amount of the compound that results in the attenuation of symptoms or a prolongation of survival in a subject. Toxicity and therapeutic efficacy of such compounds can be determined by standard pharmaceutical procedures in cell cultures or experimental animals, e.g., for determining the LD₅₀ (the dose lethal to 50% of a given population) and the ED₅₀ (the dose therapeutically effective in 50% of a given population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of a patient's condition. Dosage amount and interval can be adjusted individually to provide plasma levels of the active moiety which are sufficient to maintain the desired effects.

In case of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The pharmaceutical compositions of the present invention can be manufactured in a manner that is itself known, e.g., by means of conventional mixing, dissolving, granulating, levigating, emulsifying, encapsulating, entrapping or lyophilizing processes.

Pharmaceutical compositions for use in accordance with the present invention thus can be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention can be formulated in aqueous solutions, preferably in physiologically compatible buffers such as Hank's solution, Ringer's solution, or physiological saline buffer. For transmucosal administration, penetrants appropriate to the barriers to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinyl-pyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage for, e.g., in ampoules or in multidose containers, with an added preservatives. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspension. Suitable lipohilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations previously described, the compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

A pharmaceutical carrier for the hydrophobic compounds of the invention is a co-solvent system comprising benzyl alcohol, a non-polar surfactant, a water-miscible organic polymer, and an aqueous phase. Naturally, the proportions of a co-solvent system can be varied considerably without destroying its solubility and toxicity characteristics. Furthermore, the identity of the co-solvent components can be varied.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds can be delivered using a sustained-release system, such as semipermeable matrices of solid hydrophobic polymers containing the therapeutic agent. Various of sustained-release materials have been established and are well known to those skilled in the art. Sustained-release capsules can, depending on their chemical nature, release the compounds for a few weeks up to over 100 days. Depending on the chemical nature and the biological stability of the therapeutic reagent, additional strategies for protein stabilization can be employed.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration can, e.g., include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, e.g., via injection of the compound directly into an affected area, often in a depot or sustained release formulation.

Furthermore, one can administer the compound in a targeted drug delivery system, e.g., in a liposome coated with an antibody specific for affected cells. The liposomes will be targeted to and taken up selectively by the cells.

The compositions can, if desired, be presented in a pack or dispenser device which can contain one or more unit dosage forms containing the active ingredient. The pack can, e.g., comprise metal or plastic foil, such as a blister pack. The pack or dispenser device can be accompanied by instruction for administration. Compositions comprising a compound of the invention formulated in a compatible pharmaceutical carrier can also be prepared, placed in an appropriate container, and labeled for treatment of an indicated condition. Suitable conditions indicated on the label can include treatment of a disease such as described herein.

### EXAMPLE

### Objective

The objective of this study was to compare the efficacy cytotoxic bacteria Bordetella pertussis, administered alone or in combination with DAVANAT™ (a galactomannan, GM)/5-FU, to reduce or retard the growth of 16B-F0 melanoma tumors. The GM was processed to give a molecular weight of 50,000 Daltons with mannose to galactose ratio of 1.7.

### Test System

A total of 80 CF7BL/6N male mice were obtained from Charles River Laboratories, Inc., Portage, Mich., for the study. One animal died during the acclimation period. The mice were seven-weeks-old and weighed between 13 and 23 g at the time of tumor cell administration.

### Cancer Cell Preparation and Injection

Cells of mouse melanoma cell line, B16-F0 (ATCC CRL-6322), were placed in a flask of growth medium (DMEM+10% FBS+L-Glutamine, NEAA and penicillin/streptomycin), and were maintained at 37° C./5% CO₂/95% RH. Cells were split and maintained as such up to time of dosing.

On the day of cell administration flasks of B16-F0 cells were rinsed with warmed PBS, trypsinized, and placed in an incubator for approximately 5 minutes. The cell suspension was rinsed twice by centrifugation and resuspended in sterile saline (0.9%). The cells were counted and adjusted to a concentration of 10⁷ cells/mL and 0.1 mL was injected into the scapula area of each mouse. Mice were injected on a Tuesday. Tumor growth was evaluated each subsequent Wednesday, Friday and Monday.

### Bacterium and Test Article Dose Preparations

Each dose preparation of B. pertussis was initiated from frozen storage. Due to the rapid growth of the tumors, the first dose of the organism was prepared directly from an agar culture. The organism was grown on Bordet-Gengou agar for 3 days at 35° C., growth was transferred to PBS and concentration adjusted as necessary using a spectrophotometer. For the second dosing, the organism was grown on agar, as previously, and transferred to Stainer-Scholte broth. The broth culture was incubated on a rotary shaker at 35° C. for 2 days at which time the culture was at mid-log phase. The culture was adjusted as appropriate with sterile PBS and used for i.v. injection at a volume of 4 mL/kg. The concentration of the inoculum was verified using the dilution plate count method. The actual number of colony-forming units delivered to the animals at the two treatments times were as follows:

| Date | No. cfu/ | |
|---|---|---|
| administered | 0.1 mL | No. cfu/kg |
| Feb. 24 | 4.40 × 10⁷ | 1.76 × 10⁹ |
| Feb. 28 | 5.20 × 10⁷ | 2.10 × 10⁹ |

The test articles, GM and 5-FU, were diluted in sterile saline (0.9%) to deliver the intended dose via i.v. injection in 4 mL/kg for each article.

### Assignment to Study Groups

Animals were examined three times each week for tumor development with acknowledgement of presence/absence and size. The mean tumor size was 20.8 mg with a range from 0 to 288.0 mg. Only two animals had tumors outside of the intended 100-150 mg range. On Monday, February 23, the mean tumor size was 699.0 mg, with a range of 0 to 3967.5 mg. The animals were sorted by tumor weight and the 20 animals with the largest tumors were eliminated. The remaining 60 animals were sorted by tumor weight, largest to smallest and assigned 10 per group. The median weight and standard deviation of tumors were monitored over the next 2 weeks. Two cycle of treatment were given due to the aggressive growth of tumors. The growth rate for each group was measured and the median time (days) to reach half the size of the untreated control group was calculated:

| | | Days for Tumor to reach |
|---|---|---|
| Group# | Treatment Group | 50% of Control |
| 1 | Control PBS only | 4.833 |
| 2 | B.p. in PBS | 8.12 |
| 3 | B.p. in DAV/saline | 8.28 |
| 4 | B.p. in PBS followed by | 9.65 |
| | 5FU/DAV | |
| 5 | B.p. in DAV/saline followed by | 5.78 |
| | 5FU/DAV | |
| 6 | 5FU/DAV | 7.6 |

Notes:
Control: treatment with PBS (phosphate buffered saline) vehicle alone
B. pertussis, B.p.: treatment with log phase B. pertussis in Saline
FU/DAV: treatment with 5-fluorouracil (50 mg/Kg) co-adminstrated with galactomannan (120 mg/Kg) derivative (molecular weight 50,000 D), 24 hours post the B.p. treatment.

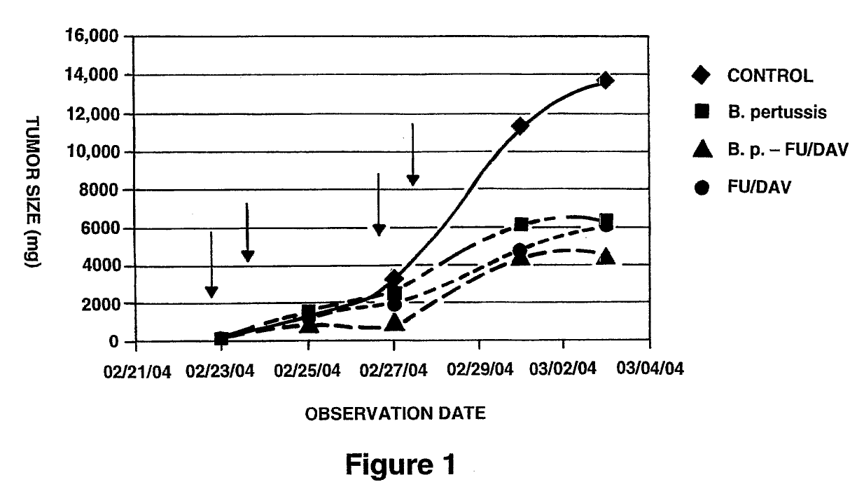

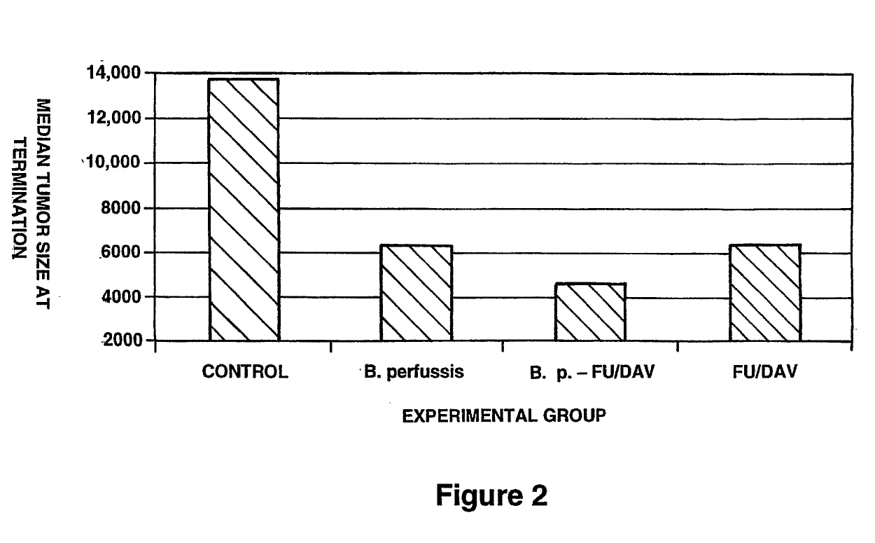

Clearly the treatment with B. pertussis followed by 5-FU combined with GM was the best of all treatments. FIGS. 1 and 2 demonstrate also the medina growth of tumors over time and the final tumor size at termination of the study.

Although the invention has been described with respect to various embodiments, it should be realized this invention is also capable of a wide variety of further and other embodiments.

What is claimed is:

1. A therapeutic composition used to treat cancer, comprising a cytotoxic microorganism and one or more chemotherapeutic agent, wherein said cytotoxic microorganism has a lectin that binds to a carbohydrate moiety on the surface of a cancer cell, and wherein said composition synergistically inhibits tumor growth.

2. The composition of claim 1, wherein said chemotherapeutic agent is selected from the group consisting of aminoglutethimide, Amsacrine, Anastrozole, asparaginase, BCG, bicalutamide, Bleomycin, Buserelin, Busulfan, Capecitabine, carboplatin, Carmustine, chlorambucil, cisplatin, Cladribine, Clodronate, cyclophosphamide, cyproterone, Cytarabine, Dacarbazine, Dactinomycin, Daunorubicin, diethylstilbestrol, Docetaxel, Doxorubicin, Epirubicin, Estramustine, etoposide, Exemestane, Filgrastim, Fludarabine, Fludrocortisone, fluorouracil, Fluoxymesterone, Flutamide, Gemcitabine, Goserelin, hydroxyurea, Idarubicin, Ifosfamide, Imatinib, Interferon Alfa, Irinotecan, Letrozole, Leucovorin, Leuprolide, Levamisole, Lomustine, Mechlorethamine, Medroxyprogesterone, Megestrol, Melphalan, mercaptopurine, Mesna, methotrexate, mitomycin, Mitotane, Mitoxantrone, Nilutamide, Octreotide, Oxaliplatin, Paclitaxel, Pamidronate, Pentostatin, Plicamycin, Porfimer, procarbazine, Raltitrexed, Rituximab, streptozocin, Tamoxifen, Temozolomide, Teniposide, testosterone, thioguanine, Thiotepa, Topotecan, Trastuzumab, Tretinoin, Vinblastine, Vincristine, Vindesine, Vinorelbine and the like.

3. The composition of claim 1, wherein said cytotoxic microorganism is selected from the group consisting of clostridium, pertussis, diptheria, listeria, and the like.

4. The composition of claim 1, wherein said composition effectuates necrosis of cancer cells.

5. A composition for the treatment of cancer, comprising an effective amount of a cytotoxic fixed microorganism capsules and a chemotherapeutic agent, wherein said composition effectuates a synergistic therapeutic effect.

6. The composition of claim 5, wherein said cytotoxic fixed microorganism has lectins that bind to carbohydrate moieties on the surface of cancer cells.

7. The composition of claim 6, wherein said carbohydrate moieties is galectin.

8. The composition of claim 7, wherein said galectin is galectin-3.

9. The composition of claim 5, wherein said composition effectuates tumor necrosis.

10. A method of treating a subject suffering from cancer by administering an effective therapeutic amount of a composition comprising a cytotoxic microorganism and one or more chemotherapeutic agents.

11. The method of claim 10, wherein said chemotherapeutic agent is selected from the group consisting of aminoglutethimide, Amsacrine, Anastrozole, asparaginase, BCG, bicalutamide, Bleomycin, Buserelin, Busulfan, Capecitabine, carboplatin, Carmustine, chlorambucil, cisplatin, Cladribine, Clodronate, cyclophosphamide, cyproterone, Cytarabine, Dacarbazine, Dactinomycin, Daunorubicin, diethylstilbestrol, Docetaxel, Doxorubicin, Epirubicin, Estramustine, etoposide, Exemestane, Filgrastim, Fludarabine, Fludrocortisone, fluorouracil, Fluoxymesterone, Flutamide, Gemcitabine, Goserelin, hydroxyurea, Idarubicin, Ifosfamide, Imatinib, Interferon Alfa, Irinotecan, Letrozole, Leucovorin, Leuprolide, Levamisole, Lomustine, Mechlorethamine, Medroxyprogesterone, Megestrol, Melphalan, mercaptopurine, Mesna, methotrexate, mitomycin, Mitotane, Mitoxantrone, Nilutamide, Octreotide, Oxaliplatin, Paclitaxel, Pamidronate, Pentostatin, Plicamycin, Porfimer, procarbazine, Raltitrexed, Rituximab, streptozocin, Tamoxifen, Temozolomide, Teniposide, testosterone, thioguanine, Thiotepa, Topotecan, Trastuzumab, Tretinoin, Vinblastine, Vincristine, Vindesine, Vinorelbine and the like.

12. The method of claim 10, wherein the microorganism is attenuated.

13. The method of claim 10, wherein said microorganism is selected from the group consisting of clostridium, pertussis, diptheria, listeria, and the like.
